(19) United States
(12) Patent Application Publication
(10) Pub. No.: US 2005/0158861 A1
(43) Pub. Date: Jul. 21, 2005

(54) Yeast transformation vector containing auxotrophic dominant gene yeast transfomant containing it and their preparation

(76) Inventor: Jae-Mahn Song, Puchon-si (KR)

Correspondence Address: LADAS & PARRY, 26 WEST 61ST STREET, NEW YORK, NY, 10023, US

(21) Appl. No.: 10/481,096

(22) PCT Filed: Jun. 20, 2002
(86) PCT No.: PCT/KR02/01179

(30) Foreign Application Priority Data
Jun. 21, 2001 (KR) 2001/35249

(51) Int. Cl. C12N 15/74; C07H 21/04; C12N 9/10; C12N 1/18; C07K 14/395

(52) U.S. Cl. 435/483; 435/193; 435/254.21; 530/350; 536/23.7

(57) ABSTRACT

A yeast transformation vector containing a dominant auxotrophic gene, a yeast transformant containing the same and a method of preparation thereof. An adenine auxotrophic transformant able to grow in the presence of adenine can be obtained by inducing a dominant adenine auxotrophic (DAD) mnutation, isolating the dominant adenine auxotrophic gene DAD1, DNA sequencing the DAD1 gene to determine its mutation site, constructing pCABIOD101 vector containing the DAD1 gene (accession number: KCTC 1013BP), and transforming haploid laboratory yeast strains and diploid industrial yeast strains respectively with the pCABIOD101 vector. Therefore, the transformation vector system of the present invention can be used in improving existing industrial yeasts and various microorganisms.

### TECHNICAL FIELD

The present invention relates to a yeast transformation vector containing a dominant auxotrophic gene, a yeast transformant containing the same and a method of preparation thereof. More particularly, the present invention relates to construction of a yeast transformation vector system by which characteristics of various microorganisms including typical industrial yeasts can be improved, comprising inducing a dominant adenine auxotrophic mutation, isolating the dominant adenine auxotrophic gene DAD1, sequencing the DAD1 gene to determine its mutation site, and constructing pCABIOD101 vector containing the DAD1 gene.

### BACKGROUND ART

Yeasts are safe for human consumption and have been used in bread, beer, distilled liquor, wine or clear strained rice wine production from the beginning of human history. They are now regarded as a GRAS (Generally Regarded As Safe) organism. Various studies of yeasts have been steadily carried out for developing industrial and commercially useful substances using it or improving characteristics of typical industrial yeasts. However, most industrial yeasts are diploid or polyploid organisms and thus difficult to mutate. Furthermore, they are sterile and thus spore formation and mating with other yeasts do not readily occur. For these reasons, satisfactory results have not been obtained in most yeast studies, unlike those using industrial bacteria. Recently, genetic recombination technologies using transformation systems for yeasts to overcome such limitations have been applied, thereby allowing development of yeasts having improved efficiency and productivity. The transformation systems for industrial yeasts, Pichia pastoris and Hansenula polymorpha have been developed and commercialized by Phillips Petroleum (Bartlesville, OK, USA) and Rhein Biotech (Dusseldorf, Germany) respectively. Recently, the transformation system for yeast Saccharomyces cerevisiae using an Aureobasidin A-resistance gene has been commercialized by Takara Shuzo (Shiga, Japan). As well, various yeast transformation vectors containing wild-type genes or antibiotic- or chemical-resistance genes have been used in laboratory yeasts. On the other hand, with respect to industrial yeasts, transformation vectors containing antibiotic- or chemical-resistance genes such as aureobasidin A, chloramphenicol, G418/geneticin, zeocin, copper, methatrexate, methylglyoxal, sulfometuron, and glyphosphate-resistance genes have mainly been used. Recently, however, where the transformants containing the above antibiotic- or chemical-resistance genes are used in industry, in particular the food industry, harmfulness to the human body by the contained resistance genes is emerging as a serious problem. Therefore, there is a need for development of new transformation vector systems for yeasts which do not require specially designed yeast host strains for yeast transformation without using existing antibiotic- or chemical-resistance genes. In this regard, the transformation vector systems for yeasts using dominant auxotrophic genes can be used in existing yeast host strains that have been used in relevant industries for a long time, thus reducing development time. Furthermore, the systems can continue to utilize existing facilities and thus are commercially available at economical prices. In addition, yeast transformants containing such yeast transformation vectors can be regarded as GRAS organisms and thus useful substances in the food industry can be produced in large quantities using them.

ADE3, one of genes that are involved in biosynthetic process of purine (adenine) in yeast S. cerevisiae is C1-tetrahydrofolate synthase gene. After mutant forms thereof were first reported by Roman (Roman, H., C.R. Lab. Carlsberg, Ser. Physiol. 26, 299-314 (1956)), a variety of mutants have been isolated by researchers to date. However, all ade3 mutants that have been reported until now are genetically recessive, like most other auxotrophic mutants. That is, haploid ade3 yeasts are adenine auxotrophs, but once they become diploid ade3/ADE3⁺ yeasts, they do not require supplementary adenine any more. For this reason, recessive auxotrophic genes cannot be used as selectable genes in yeast transformation vectors.

### DISCLOSURE OF THE INVENTION

Therefore, the present inventor induced a dominant adenine auxotrophic mutation in the ADE3 gene using genetic recombination techniques, not conventional gene assay protocols, and then sequenced the mutated ADE3 gene site. The dominant adenine auxotrophic mutant gene is a special gene, from a molecular biological point of view, among all microorganisms including yeasts, and therefore was designated DAD1 Dominant adenine-requiring) gene. That is, both diploid DAD1/dad1⁺ yeasts and haploid DAD1 yeasts are adenine auxotrophs. The DAD1 gene can be used in elucidating roles of its gene product, C1-tetrahydrofolate synthase in a purine synthesis process, and moreover, its industrial significance and merits are considerable when used in biological industry.

Therefore, an object of the present invention is to construct a yeast transformation vector, comprising inducing a dominant adenine auxotrophic mutation, isolating and sequencing the dominant adenine auxotrophic gene, and constructing the yeast transformation vector containing the dominant adenine auxotrophic gene. Another object of the present invention is to provide an adenine auxotrophic transformant containing the yeast transformation vector.

In accordance with the present invention, the above and other objects can be accomplished by construction of an adenine auxotrophic transformant able to grow in the presence of adenine, comprising inducing a dominant adenine auxotrophic mutation in yeast C1-tetrahydrofolate synthase gene ADE3 using hydroxylamine, isolating the dominant adenine auxotrophic gene DAD1, DNA sequencing the DAD1 gene to determine its mutation site, constructing pCABIOD101 vector containing the DAD1 gene, and transforming haploid yeast Saccharomyces cerevisiae and diploid industrial yeast strains respectively with the pCABIOD101 vector.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flow diagram showing the process for constructing the final transformant in accordance with the present invention;

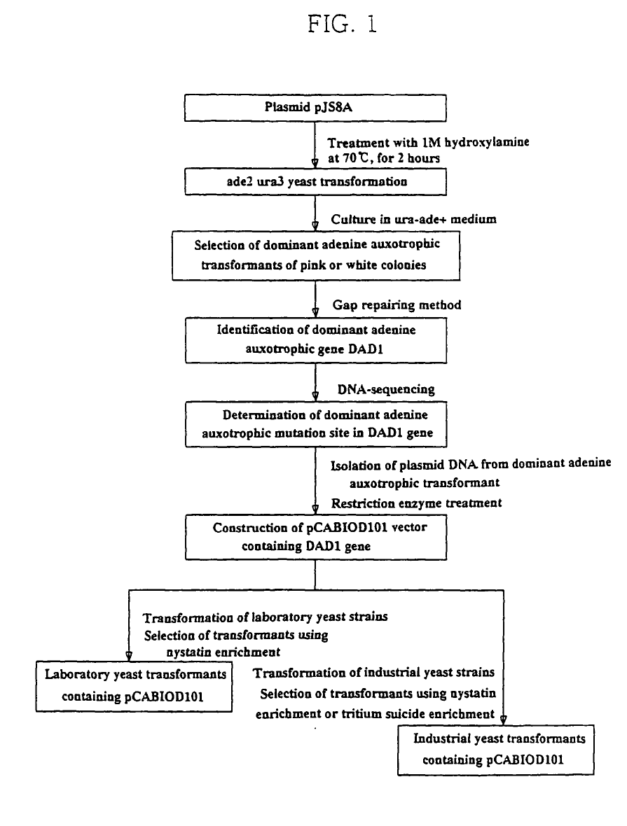

FIG. 2 is a view showing plasmid deletions for determining the dominant adenine auxotrophic mutation site in DAD1 gene; and

FIG. 3 is a restriction map of pCABIOD101 vector containing the dominant adenine auxotrophic gene DAD1.

### BEST MODE FOR CARRYING OUT THE INVENTION

Hereinafter, the constitutional elements of the present invention will be described in more detail.

The present invention is accomplished by carrying out following steps: inducing a dominant adenine auxotrophic mutation in yeast C1-tetrahydrofolate synthase gene; isolating the dominant adenine auxotrophic gene; DNA sequencing the dominant adenine auxotrophic gene to determine its mutation site; constructing a vector containing the dominant adenine auxotrophic gene; and transforming haploid yeast Saccharomyces cerevisiae and diploid industrial yeast strains respectively with the vector and then selecting adenine auxotrophic transformants.

Plasmid pJS8A containing yeast ADE3 gene and URA3 gene used in the present invention is that disclosed in Curr. Genet, 16:315-321.

The present invention will hereinafter be described more specifically by illustrative examples. It is, however, to be borne in mind that the present invention is by no means limited to or by them.

### EXAMPLE 1:

### Construction of Yeast Transformation Vector Containing Yeast Dominant Adenine Auxotrophic Gene

Step 1: Induction of Dominant Adenine Auxotrophic (DAD) Mutation (Construction of DAD Yeast Strain)

Plasmid pJS8A containing yeast ADE3 and URA3 genes (Song, J. M. and Liebman, S. W., Curr. Genet. 16, 315-321) was treated with 1M hydroxylamine at 70° C. for 2 hours, causing in vitro mutation. Then, ade2 ura3 yeast S. cerevisiae was transformed with the plasmid and cultured in a small amount of adenine (5.0 μg/ml)-containing medium without uracil, thereby producing Ura⁺ transformants, which formed red colonies except for one pink colony. This is because when genes (for example ADE3) epistatic to ade2 are mutated in the adenine biosynthesis pathway, formation of red pigment is prevented by blocking of the biosynthesis of adenine due to the accumulation of the ade2 product (CAIR, 5-amino4-imidazolecarboxylate ribonucleotide). Pink or white colonies are formed depending on degree of blocking of adenine biosynthesis. Therefore, the pink transformant may be that transformed with the plasmid containing a dominant adenine auxotrophic mutation. For this reason, the plasmid DNA was isolated from the transformant using Escherichia coli and digested with restriction enzymes BamHI and SalI, thereby to obtain a 6.7 kb DNA fragment containing the ADE3 gene. Then, the obtained DNA fragment was transfected into ade3-130 yeast strain to thereby give a transformant.

The above transformant containing the dominant adenine auxotrophic mutation was designated as Saccharomyces cerevisiae S68. It was deposited in Genetic Resources Center, Korea Research Institute of Bioscience and Biotechnology on May 31, 2001 under KCTC 1018BP.

Diploid yeast strains also maintain adenine auxotrophism, like in the above yeast strain. Therefore, the adenine auxotrophism is dominant. Furthermore, dominant adenine auxotrophism is more strongly manifested at a low temperature of 23° C. or less. The phenotypes of the above dominant adenine auxotrophic mutants are presented in Table 1 below. Yeast suspensions were inoculated by spotting in synthetic complete media (SC) and adenine deficient media (SC-Ade) respectively, followed by comparison of growth characteristics.

TABLE 1
Phenotypes of dominant adenine auxotrophic mutants

| | |
|---|---|
| SC* | SC-Ade* |

| Partial genotype | 30° C. | 23° C. | 30° C. | 23° C. |
|---|---|---|---|---|
| ADE3* (or dad1*) | + | + | + | + |
| Ade3-130 | + | + | − | − |
| DAD1 | + | + | ± − | − |
| DAD1/dad1* | + | + | ± | ± − |

*Yeast suspensions were inoculated by spotting in synthetic complete media (SC) and adenine deficient media (SC-Ade) respectively, followed by comparison of growth characteristics.
The evaluation of yeast growth is as follows:
+, good growth by 1 or 2 days;
± , some growth by 4 days;
± −, some growth by 7 days;
−, no sign of growth by 7 days.

Step 2: Identification of Dominant Adenine Auxotrophic Gene (DAD1 )

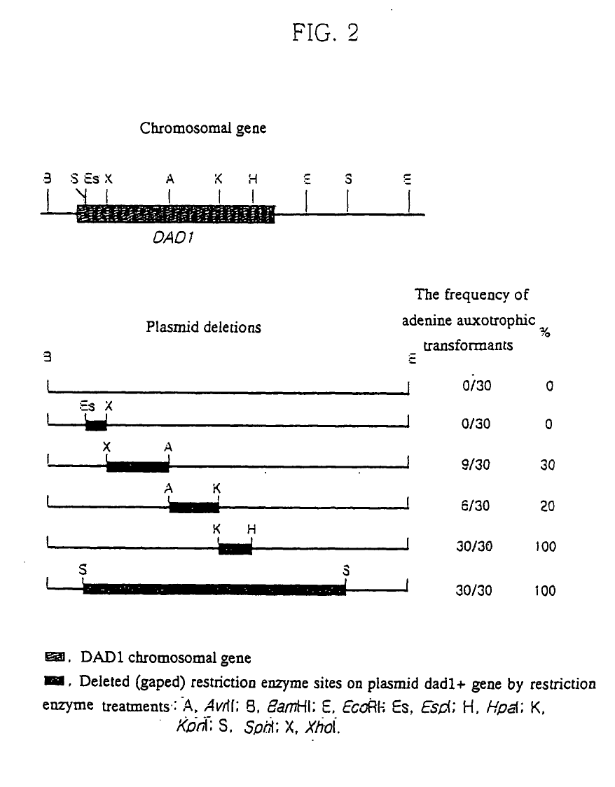

The dominant adenine auxotrophic yeast strain obtained in step 1 was transformed with multicopy plasmid YRpADE3 in which ADE3⁺, URA3⁺ and ARS1 genes were contained, thereby to obtain Ura⁺ transformants. The Ura⁺ transformants were able to grow in the absence of adenine. This fact indicates that dominance of one chromosomal dominant gene is masked by the wild type gene of the multicopy plasmid. Based on the above fact, the plasmid YRpADE3 was treated with respective combinations of EspI and XhoI, XhoI and AvrII, AvrII and KpnI, KpnI and HpaI, SphI and SphI to thereby obtain gap repaired YRpADE3 plasmids. Dominant adenine auxotrophic yeast strains were transformed with the gap repaired YRpADE3 plasmids, producing Ura⁺ transformants. Upon investigating the adenine auxotrophism of each of the Ura⁺ transformants, the transformants obtained using the KpnI and HpaI restriction enzymes- or the SphI and SphI restriction enzymes-treated plasmid exhibited adenine auxotrophism. From the above result, it can be seen that a dominant adenine auxotrophic mutation site is present between a KpnI and an HpaI restriction enzyme site in C1-tetrahydrofolate synthase gene, as shown in FIG. 2. Therefore, the C1-tetrahydrofolate synthase gene containing the dominant adenine auxotrophic mutation site was designated as DAD1 (dominant adenine-requiring) gene.

Step 3: Determination of Dominant Adenine Auxotrophic (DAD) Mutation Site

The 497 bp DNA sequence of the KpnI-HpaI restriction fragment containing the dominant adenine auxotrophic mutation site identified in step 2 was sequenced by DNA-sequencing technique using Sequenase Version 2.0 DNA sequencing kit (Biochemical Corp. USA). The DNA sequence of DAD1 gene is set forth in SEQ ID NO: 1. The amino acid sequence encoded by the DAD1 gene is set forth in SEQ ID NO: 2. The DAD mutation was a point mutation to the base sequence of codon 683 among the total 947 codons in yeast C1-tetrahydrofolate synthase gene (TTC was changed to TCC), resulting in altering the corresponding amino acid from phenylalanine to serine.

Step 4: Construction of Plasmid pCABIOD101 Containing DAD1 Gene

The KpnI and HpaI restriction enzymes-treated plasmid DNA in step 2 was isolated from the dominant adenine auxotrophic transformant containing the plasmid using E. coli and treated with NheI restriction enzyme. The NheI restriction enzyme-treated plasmid DNA was treated with Klenow enzyme to give blunt ends, and then with BamHI restriction enzyme to give a 3.68 kb DNA fragment, which was substituted for 406 bp BamHI-SmaI restriction enzyme site of yeast insertion vector pJS41. As a result, a new yeast insertion plasmid vector containing the dominant adenine auxotrophic gene DAD1 was constructed.

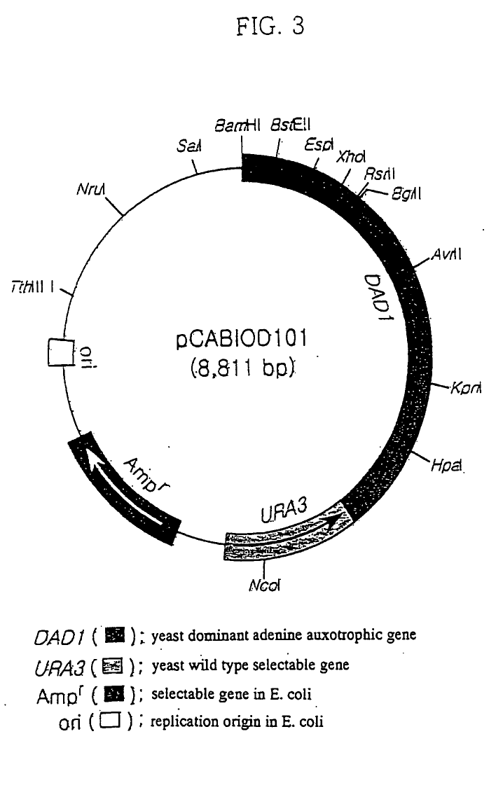

The above vector was designated as pCABIOD101. It was deposited in Genetic Resources Center, Korea Research Institute of Bioscience and Biotechnology on May 28, 2001 under KCTC 1013BP. The restriction map of the vector pCABIOD101 is shown in FIG. 3.

The pCABIOD101 plasmid is an insertion vector that ensures inserted genes to be stably maintained in yeast chromosomal DNA without self-replicating in yeasts. After one of the single restriction enzyme sites (AvrII, BglII, BstEII, EspI, RsrII, SacII, Xhol) in the DAD1 gene of the plasmid was digested, the plasmid was inserted into the dad1⁺ gene of yeast chromosomal DNA. The yeast transformant obtained in the above manner contains single- or multi-copy plasmid in its chromosome and inserted genes can be stably maintained in the transformant. The pCABIOD101 plasmid contains another yeast selectable gene URA3⁺ in addition to the DAD1 gene and thus can be readily utilized in transformation of (uracil) auxotrophic ura3 yeast strain that is now widely used as laboratory yeast.

### EXAMPLE 2:

### Transformation of S. cerevisiae with the pCABIOD101 Vector

The plasmid pCABIOD101 constructed in step 4 was treated with restriction enzyme BstEII to obtain linearized DNA and was integrated into S. cerevisiae GT48 (a ade3-130 ser1-171 ura3-52) and S73 (a ser1-171 ura3-52) respectively. Then, adenine auxotrophic transformants were selected using nystatin enrichment. For the nystatin enrichment, the transformed yeasts were plated on 0.4 ml minimal media supplemented with amino acids and ammonium sulfate which were required for growth of host strains and transformants, and then cultured at 30° C., at 300 rpm, for 6 hours. The cultured yeast cells were washed with sterile distilled water, plated on 0.4 ml ammonium sulfate-free minimal media, and cultured at 30° C., at 300 rpm, for 12 to 14 hours, thereby inducing nitrogen starvation. The growth-suspended cells due to nitrogen starvation were washed with sterile distilled water and plated on 0.36 ml minimal media supplemented with amino acids and ammonium sulfate which were required for growth of host strains. The minimal medium-treated cells were cultured at 23 ° C., at 300 rpm for 6 hours. 25 to 70 μl of 30 μg/ml nystatin solution was added to the yeast cultures in which the host strains had begun to grow, at 23 ° C., at 300 rpm for 1 hour. The nystatin treated cells were washed with sterile distilled water and then the above process was once again repeated except for treatment with nystatin for 1 hour 30 minutes. The nystatin treated cells were suspended in sterile distilled water, plated on selectable media (adenine deficient media, SC-Ade) and cultured at a low temperature of 23 ° C. or less, thereby selecting slowly growing small-sized colonies as transformants. To determine whether the selected small-sized colonies were the desired transformants, the selected transformants were inoculated in adenine deficient media (SC-Ade) and uracil deficient media (SC-Ura) respectively by spotting method and then their growth characteristics was compared. As a control, pJS41 vector was treated with restriction enzyme NcoI to give linearized DNA and inserted into S. cerevisiae. Then, transformants were selected in selectable media (uracil deficient media, SC-Ura). The results are presented in Table 2 below.

TABLE 2
Phenotypes of laboratory yeast transformants

| | | SC** | | SC − Ade** | | SC − Ura** | |
|---|---|---|---|---|---|---|---|
| Strain [plasmid]* | | 30° C. | 23° C. | 30° C. | 23° C. | 30° C. | 23° C. |
| GT48 | [None] | + | + | − | − | − | − |
| | [pJS41] | + | + | − | − | + | + |
| | [pCABIOD101] | + | + | ±− | − | + | + |
| S73 | [None] | + | + | + | + | − | − |
| | [pJS41] | + | + | + | + | + | + |
| | [pCABIOD101] | + | + | ± | ±− | + | + |

*Yeast strains were GT48 (α ade3-130 ser1-171 ura3-52) and S73 (α ser1-171 ura3-52), and plasmids pJS41 and pCABIOD101 were inserted after treated with the restriction enzymes NcoI and BstEII respectively.
**Yeast suspensions were inoculated by spotting in synthetic complete media (SC), adenine deficient media (SC − Ade) and uracil deficient media (SC − Ura) respectively, followed by comparison of growth characteristics.
The evaluation of yeast growth is as follows:
+, good growth by 1 or 2 days;
±, some growth by 4 days;
±−, some growth by 7 days;
−, no sign of growth by 7 days.

### Example 3:

### Transformation of Industrial Yeast Strains with pCABIOD101 Vector

In the example, the plasmid pCABIOD101 constructed in step 4 of example 1 was treated with restriction enzyme BglII to give linearized DNA and inserted into an industrial yeast strain, bread yeast (diploid). Then, adenine auxotrophic transformants were selected using the nystatin enrichment used in example 2 or another auxotroph enrichment, tritium suicide enrichment. With respect to the tritium suicide enrichment, the transformed yeast culture was inoculated in 10 ml minimal medium (7 g/l amino acid-free YNB (yeast nitrogen base), 10 g/l dextrose) supplemented with adenine until 0.1 O.D₅₅₀, and then cultured at 30° C., at 300 rpm, for 5 hours. 1 ml culture was taken and transferred into screw-cap tube. 25 μ Ci/ml of ³H-sodium formate [³H] was added to the culture for labeling with tritium. Tritium-labeled cells were cultured at 23 ° C., at 300 rpm and O.D₅₅₀ values were measured at constant time intervals. The O.D₅₅₀ value did not increase after 4 hours 30 minutes, i.e. the number of cells did not increase any more. At this time, the labeled culture was treated with ice thereby to discontinue labeling. The labeled cells were washed twice with sterile distilled water, resuspended in 1 ml 1X YNB (0.7% YNB without amino acids) and stored at 4 ° C. Cells grow slowly and tritium suicide begins at 4 ° C. A designated number of cells was inoculated in YPD medium once every two days to isolate survivors. The selecting was discontinued at day 16 when cell viability is less than 10% due to tritium suicide. The tritium suicide treated cells were suspended in sterile distilled water, plated on selectable medium (adenine deficient medium, SC-Ade) and cultured at a low temperature of 23 ° C. or less, thereby to select slowly growing small-sized colonies as transformants. To determine whether the selected small-sized colonies were the desired transformnants, the selected transformants were inoculated in synthetic minimal media (SD, synthetic dextrose) and adenine-containing media (SC+Ade) respectively by spotting method and then their growth characteristics was compared. The results are presented in Table 3 below. Furthermore, PCR confirmed that the selected transformants contained the plasmid pCABIOD101.

TABLE 3
Phenotypes of bread yeast transformants

| | | SD + Ade | | SD | |
|---|---|---|---|---|---|
| Strain [plasmid] | | 30° C. | 23° C. | 30° C. | 23° C. |
| Bread yeast | [None] | + | + | + | + |
| | [pCABIOD101] | + | + | ± | ±− |

The evaluation of yeast growth is as follows:
+, good growth by 1 or 2 days;
±, some growth by 4 days;
±−, some growth by 7 days;
−, no sign of growth by 7 days.

### INDUSTRIAL APPLICABILITY

As apparent from the above description, the present invention provides the construction of an adenine auxotrophic transformant, comprising inducing a dominant adenine auxotrophic (DAD) mutation, isolating the dominant adenine auxotrophic gene DAD1, DNA sequencing the DAD1 gene to determine its mutation site, constructing pCABIOD101 vector containing the DAD1 gene, and transforming haploid laboratory yeasts and diploid industrial yeasts respectively with the pCABIOD101 vector. Therefore, the transformation vector system of the present invention can be used in improving existing industrial yeasts and various microorganisms and thus is an industrially useful invention.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

[image]

1. A method for constructing a dominant adenine auxotrophic Saccharomyces cerevisiae S68 strain (accession number: KCTC 1018BP), comprising the steps of:
inducing in vitro mutation by treating plasmid pJS8A containing yeast ADE3 and URA3 genes with hydroxylamine;
transforming ade2 ura3 yeasts with the plasmid pJS8A and culturing the transformants in -Ura± Ade medium;
isolating plasmid DNA from pink or white transformant colonies containing mutations to the ADE3 gene epistatic to ade2 using E. coli; and
treating the plasmid DNA with restriction enzymes BamHI and SalI to isolate a DNA fragment containing the mutated ADE3 gene and transfecting the DNA fragment into yeast strain ade3-130.

2. A dominant adenine auxotrophic DAD1 gene set forth in SEQ ID NO: 1.

3. An amino acid sequence set forth in SEQ ID NO: 2 encoded by the dominant adenine auxotrophic DAD1 gene set forth in SEQ ID NO: 1.

4. A pCABIOD101 recombinant vector containing the dominant adenine auxotrophic DAD1 gene set forth in SEQ ID NO: 1 (accession number: KCTC 1013BP).

5. A yeast transformant, Saccharomyces cerevisiae pCABIOD101 constructed by transforming yeast Saccharomyces cerevisiae with the pCABIOD101 recombinant vector as set forth in claim 4.

6. A method for selecting dominant adenine auxotrophic transformants containing pCABIOD101 recombinant vectors (accession number: KCTC 1013BP) using tritium suicide enrichment, in which ³H-sodium formate [³H] is used as tritium in the tritium suicide enrichment.


SEQUENCE LISTING (2 sequences)

SEQ ID NO: 1 - DNA, 2841 bp, Saccharomyces cerevisiae
  CDS (1)..(2838): Dominant adenine-requiring transformed saccharomyces (DAD)
atggctggtcaagtgttggacggcaaagcatgcgctcagcagtttagaagcaatattgctaatgaaatcaaaagcattcaaggtcacgtgcctgggtttgcacctaaccttgccatcattcaagtaggcaacagaccagactcagccacatatgtacgcatgaagcgtaaggcagctgaagaggccggcattgttgctaatttcattcatttagatgaatccgctactgaatttgaagttctgcgttacgtggaccagctgaatgaggacccacatacacacggtattatcgtgcaactaccattacccgctcatttggacgaggatagaatcacctcgagagtgttggcagaaaaggacgtggacgggttcgggcccaccaacattggcgaattgaataagaagaacggacacccattctttttgccctgcacgcccaaggggatcattgagctgcttcacaaggccaacgtcacgattgaaggttcccggtccgttgtgatcggaagatctgacattgttggctctcctgttgcagaattgttaaaatctctaaactccaccgtcaccatcactcattctaaaacccgtgatatcgcatcatacttacacgacgcggacatcgtagtcgttgccatcggccaaccagaatttgtgaagggtgaatggttcaaaccaagagacggcacttccagtgataagaaaaccgtggtaattgatgttggcaccaactacgttgctgatccttccaaaaagtccggtttcaaatgtgttggtgacgttgagttcaatgaagcaatcaaatacgtccatctaatcactccagtgcccggtggtgtgggccccatgacggtggctatgttaatgcaaaataccttgattgctgccaaacgccaaatggaagaatcctcgaagcctttgcagattcctcccttgccattgaagttgctaacacctgttccttccgatatagacatctccagagcacaacagccaaagcttatcaaccagcttgctcaagaattgggtatttactctcatgagttggagctgtacggacattacaaggccaaaatttctcctaaagtcatcgaaaggctgcagacgcgccaaaatggtaagtacatcttggtgtctggtatcacaccaacaccactgggagagggtaaatccactacaacaatgggtcttgtccaggcactaacggctcacttgggcaagccagccattgcgaacgtcagacaaccctccctaggacccactttaggtgtcaaaggtggtgctgcgggtggtggttattcccaagtcatcccaatggacgaattcaacttacatttgactggtgacattcacgccattggtgcggctaacaacctacttgctgccgctattgacactagaatgttccatgagaccactcaaaagaacgacgctaccttctacaacagactagtgcctagaaagaacggaaagagaaagtttactccctccatgcaaagaagattgaacagactgggtattcaaaagaccaaccccgatgatctaacacccgaagagatcaacaaattcgccagattgaacattgacccggacactattactatcaagagggtggtcgatatcaacgacagaatgttaagacaaatcaccattggtcaagcccctaccgagaagaaccacacaagagttactggattcgatatcaccgttgcttctgaattgatggctattcttgctctttcaaaggacttgagggacatgaaggaacgtattggaagagtcgttgttgctgctgacgtaaacaggtctccagtcactgttgaagatgtgggttgtaccggtgccttaaccgctttattaagagacgctatcaagcccaacttgatgcaaactttagaaggtactcctgtcttggtccatgccggcccatttgccaacatctctatcggtgcctcttctgttattgctgatcgcgtggctttgaaattggttggtaccgagccagaggcaaaaacagaagctggttatgtggttactgaagcagggtccgatttcactatgggtggtgaaagattcttcaacatcaagtgccgttcctctggattgacacctaatgctgtggtcttggttgctactgttagggcattgaagtcacacggtggtgctccagatgtcaaacctggccaacctttaccttccgcatacactgaagagaatatcgagtttgtcgaaaaaggtgccgctaacatgtgtaaacaaattgccaacattaagcaatttggcgtccccgtcgttgtcgcaattaacaagtttgaaactgacactgaaggtgaaatagccgccattagaaaagccgctttggaagctggtgcatttgaagccgtaacctctaaccattgggccgaaggtggtaaaggtgctatcgacttggccaaggccgtcatcgaagcttccaaccaaccagtggacttccatttcctatatgacgttaactcctccgttgaagacaaattaactactatcgttcaaaagatgtacggtggtgccgcaatcgatatcttgcctgaagcacaacgcaagattgacatgtacaaggaacaaggtttcggtaacttgccaatttgtatcgccaagacacaatactctttatcccacgatgcaactttgaaaggtgttccaaccgggttcactttccccatcagagacgtcagattgtctaatggtgctggatacttatacgctcttgccgccgaaatacaaaccattcctggtttggctacctatgctggttacatggccgtggaagtcgatgatgacggtgagatcgatggcctgttctaa

SEQ ID NO: 2 - protein, 946 aa, Saccharomyces cerevisiae
  Dominant adenine-requiring transformed saccharomyces (DAD)
MAGQVLDGKACAQQFRSNIANEIKSIQGHVPGFAPNLAIIQVGNRPDSATYVRMKRKAAEEAGIVANFIHLDESATEFEVLRYVDQLNEDPHTHGIIVQLPLPAHLDEDRITSRVLAEKDVDGFGPTNIGELNKKNGHPFFLPCTPKGIIELLHKANVTIEGSRSVVIGRSDIVGSPVAELLKSLNSTVTITHSKTRDIASYLHDADIVVVAIGQPEFVKGEWFKPRDGTSSDKKTVVIDVGTNYVADPSKKSGFKCVGDVEFNEAIKYVHLITPVPGGVGPMTVAMLMQNTLIAAKRQMEESSKPLQIPPLPLKLLTPVPSDIDISRAQQPKLINQLAQELGIYSHELELYGHYKAKISPKVIERLQTRQNGKYILVSGITPTPLGEGKSTTTMGLVQALTAHLGKPAIANVRQPSLGPTLGVKGGAAGGGYSQVIPMDEFNLHLTGDIHAIGAANNLLAAAIDTRMFHETTQKNDATFYNRLVPRKNGKRKFTPSMQRRLNRLGIQKTNPDDLTPEEINKFARLNIDPDTITIKRVVDINDRMLRQITIGQAPTEKNHTRVTGFDITVASELMAILALSKDLRDMKERIGRVVVAADVNRSPVTVEDVGCTGALTALLRDAIKPNLMQTLEGTPVLVHAGPFANISIGASSVIADRVALKLVGTEPEAKTEAGYVVTEAGSDFTMGGERFFNIKCRSSGLTPNAVVLVATVRALKSHGGAPDVKPGQPLPSAYTEENIEFVEKGAANMCKQIANIKQFGVPVVVAINKFETDTEGEIAAIRKAALEAGAFEAVTSNHWAEGGKGAIDLAKAVIEASNQPVDFHFLYDVNSSVEDKLTTIVQKMYGGAAIDILPEAQRKIDMYKEQGFGNLPICIAKTQYSLSHDATLKGVPTGFTFPIRDVRLSNGAGYLYALAAEIQTIPGLATYAGYMAVEVDDDGEIDGLF